(19) United States
(12) Patent Application Publication
(10) Pub. No.: US 2014/0199252 A1
(43) Pub. Date: Jul. 17, 2014

(54) SILICONE-FREE ANTIPERSPIRANT COMPOSITIONS AND METHODS FOR MANUFACTURING SILICONE-FREE ANTIPERSPIRANT COMPOSITIONS

(71) Applicant: The Dial Corporation, Scottsdale, AZ (US)

(72) Inventors: Travis T. Yarlagadda, Lenexa, KS (US); Haley Austin, Gilbert, AZ (US)

(73) Assignee: The Dial Corporation, Scottsdale, AZ (US)

(21) Appl. No.: 14/216,726

(22) Filed: Mar. 17, 2014

Related U.S. Application Data
(62) Division of application No. 13/349,760, filed on Jan. 13, 2012, now abandoned.

(51) Int. Cl. A61K 8/31; A61Q 15/00

(52) U.S. Cl. CPC A61K 8/31; A61Q 15/00
USPC 424/66

(57) ABSTRACT

Silicone-free antiperspirant compositions and methods for fabricating silicone-free antiperspirant compositions are provided. In accordance with an exemplary embodiment, an antiperspirant composition includes an active antiperspirant compound, stearyl alcohol, a C14-C16 fatty alcohol, and a carrier fluid. The carrier fluid is composed of a first volatile hydrocarbon component selected from a C12-C14 hydrocarbon, a second volatile hydrocarbon selected from a C13-C16 hydrocarbon, and at least one non-volatile hydrocarbon component.

### TECHNICAL FIELD

The present invention generally relates to antiperspirant compositions and methods for manufacturing antiperspirant compositions, and more particularly relates to silicone-free antiperspirant compositions and methods for manufacturing silicone-free compositions.

### BACKGROUND

Antiperspirants are popular personal care products used to prevent or eliminate perspiration and body odor caused by perspiration. Antiperspirants typically prevent the secretion of perspiration by blocking or plugging sweat-secreting glands, such as those located at the underarms. Antiperspirant solid sticks are desired by a large majority of the population because of the presence of active antiperspirant compounds that block or prevent the secretion of perspiration and its accompanying odors and because of their ease of application. A solid antiperspirant product is applied to the skin by swiping or rubbing the stick across the skin, typically of the underarm.

Antiperspirant compositions often include volatile silicones, such as cyclomethicone (also known as cyclopentasiloxane), as a hydrophobic carrier fluid to provide smooth, dry spreadability during application. However, concerns have been raised regarding the ecological and environmental impact of cyclomethicone and other volatile silicones used in cosmetics. In fact, similar silicone compounds such as cyclotetrasiloxanes have largely been removed from cosmetic use already.

Accordingly, it is desirable to provide antiperspirant compositions that contain substantially no silicone. In addition, it is desirable to provide methods for manufacturing silicone-free antiperspirant compositions. Furthermore, other desirable features and characteristics will become apparent from the subsequent detailed description and the appended claims, taken in conjunction with the accompanying drawings and this background.

### BRIEF SUMMARY

Silicone-free antiperspirant compositions and methods for fabricating silicone-free antiperspirant compositions are provided. In accordance with an exemplary embodiment, an antiperspirant composition includes an active antiperspirant compound, stearyl alcohol, a C14-C16 fatty alcohol, and a carrier fluid. The carrier fluid is composed of a first volatile hydrocarbon component selected from a C12-C14 hydrocarbon, a second volatile hydrocarbon selected from a C13-C16 hydrocarbon, and at least one non-volatile hydrocarbon component.

In accordance with another exemplary embodiment, an antiperspirant composition includes an active antiperspirant compound, stearyl alcohol present in an amount of from about 15.0 to about 22.0 weight percent of the composition, cetyl alcohol present in an amount of from about 0.3 to about 6.0 weight percent of the composition, and a silicone-free carrier fluid present in an amount of from about 25.0 to about 50.0 weight percent of the composition. The silicone-free carrier fluid is composed of a first volatile hydrocarbon component selected from a C12-C14 hydrocarbon, a second volatile hydrocarbon component selected from a C13-C16 hydrocarbon, and a non-volatile hydrocarbon component.

In accordance with an exemplary embodiment, a method for fabricating a silicone-free antiperspirant composition combines a C14-C16 fatty alcohol and stearyl alcohol in a C14-C16 fatty alcohol:stearyl alcohol ratio of from about 1:2.5 to about 1:60. The stearyl alcohol and the C14-C16 fatty alcohol are melted. In the method, a carrier fluid is added to the stearyl alcohol and the C14-C16 fatty alcohol. The carrier fluid is composed of a first volatile hydrocarbon component selected from a C12-C14 hydrocarbon, a second volatile hydrocarbon component selected from a C13-C16 hydrocarbon, and a non-volatile hydrocarbon component. The method forms a melted mixture, and pours the melted mixture into a mold. The melted mixture is allowed to cool to ambient temperature.

### BRIEF DESCRIPTION OF THE DRAWING

The present invention will hereinafter be described in conjunction with the following drawing FIGURE, wherein:

FIG. 1 is a perspective view of a silicone-free antiperspirant composition in accordance with an exemplary embodiment.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The various embodiments contemplated herein relate to a silicone-free antiperspirant composition. As used herein, the term “silicone” means any polymer comprising silicon, carbon, hydrogen, and oxygen. Further, as used herein, the term “silicone-free” means comprising no silicone or comprising silicone in an amount that does not provide a perceivable chemical or mechanical effect, whether microscopic or macroscopic, to the antiperspirant composition (or to the carrier fluid) compared to antiperspirant compositions (or carrier fluids) without silicone. For example, the antiperspirant composition (or carrier fluid) may contain no more than 5.0 weight percent silicones relative to the total antiperspirant composition (or total carrier fluid). To provide similar characteristics to conventional antiperspirants using silicones, various embodiments herein comprise a silicone-free carrier fluid that is composed of a first volatile hydrocarbon component selected from a C12-C14 hydrocarbon, a second volatile hydrocarbon component selected from a C13-C16 hydrocarbon, and a non-volatile hydrocarbon component. The substantially silicone-free carrier fluid provides volatility similar to that provided by volatile silicones and feels similar to cosmetics using volatile silicones.

Further, it is noted that various embodiments of the silicone-free antiperspirant compositions include stearyl alcohol as a desired structurant but do not exhibit visible surface crystals upon manufacture. In this regard, the various embodiments include a C14-C16 fatty alcohol, such as cetyl alcohol. While stearyl alcohol is a desired structurant in antiperspirant compositions because of its ability to impart structure and hardness to the compositions, in certain compositions it may cause visible crystals to form at the surface of the compositions after manufacture. It is believed that the C14-C16 fatty alcohol present in sufficient amounts of the silicone-free antiperspirant composition, forms a fatty alcohol matrix with the stearyl alcohol that prevents the visible crystals from forming.

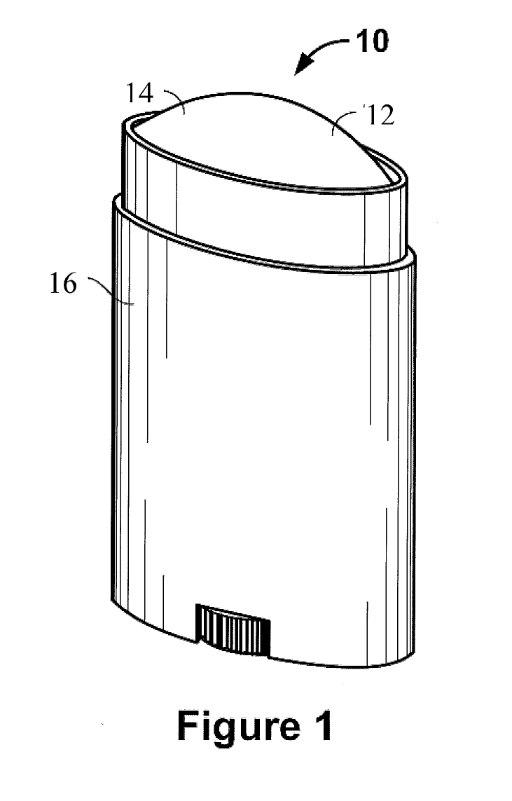

In one exemplary embodiment, the silicone-free antiperspirant composition contemplated herein is a uniform solid stick, such as an invisible solid, as shown in FIG. 1. The illustrated silicone-free antiperspirant product 10 includes a silicon-free antiperspirant composition 12. Silicone-free antiperspirant composition 12 has an application surface 14 that is substantially dome-shaped and that is configured to be applied to skin, such as, for example, an underarm. Further, silicone-free antiperspirant composition 12 is disposed in a container or dispenser 16 for dispensing silicone-free antiperspirant composition 12 to the skin.

The various embodiments of the silicone-free antiperspirant composition contemplated herein comprise a water-soluble active antiperspirant compound. Active antiperspirant compounds contain at least one active ingredient, typically metal salts, that are thought to reduce perspiration by diffusing through the sweat ducts of apocrine glands (sweat glands responsible for body odor) and hydrolyzing in the sweat ducts, where they combine with proteins to form an amorphous metal hydroxide agglomerate, plugging the sweat ducts so perspiration cannot diffuse to the skin surface. Some active antiperspirant compounds that may be used in the first and/or second portions include astringent metallic salts, especially inorganic and organic salts of aluminum, zirconium, and zinc, as well as mixtures thereof. Particularly preferred are aluminum-containing and/or zirconium-containing salts or materials, such as aluminum halides, aluminum chlorohydrates, aluminum hydroxyhalides, zirconyl oxyhalides, zirconyl hydroxyhalides, and mixtures thereof. Exemplary aluminum salts include those having the general formula Al₂(OH)ₐCl_{b}x(H₂O), wherein a is from 2 to about 5; the sum of a and b is about 6; x is from about 1 to about 6; and wherein a, b, and x may have non-integer values. Exemplary zirconium salts include those having the general formula ZrO(OH)₂₋ₐClₐx (H₂O), wherein a is from about 1.5 to about 1.87, x is from about 1 to about 7, and wherein a and x may both have non-integer values. Particularly preferred zirconium salts are those complexes that additionally contain aluminum and glycine, commonly known as ZAG complexes. These ZAG complexes contain aluminum chlorohydroxide and zirconyl hydroxy chloride conforming to the above-described formulas. Examples of active antiperspirant compounds suitable for use in the various embodiments contemplated herein include aluminum dichlorohydrate, aluminum zirconium octachlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum zirconium trichlorohydrate, aluminum zirconium tetrachlorohydrate, aluminum zirconium pentachlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, zirconium chlorohydrate, aluminum chloride, aluminum sulfate buffered, and the like, and mixtures thereof. In a preferred embodiment, the antiperspirant compound is aluminum zirconium trichlorohydrate GLY. In another embodiment, the silicone-free antiperspirant composition comprises an active antiperspirant compound present in the amount of 10.0 to about 25.0 weight percent (USP). As used herein, weight percent (USP) or wt. % (USP) of an antiperspirant salt is calculated as anhydrous weight percent in accordance with the U.S.P. method, as is known in the art. This calculation excludes any bound water and glycine.

The silicone-free antiperspirant composition further comprises a hydrophobic carrier fluid. The carrier fluid is a stable, homogeneous mixture combining multiple hydrocarbons of different structures. Specifically, the carrier fluid is composed of a first volatile hydrocarbon component, a second volatile hydrocarbon component, and a non-volatile hydrocarbon component. The volatile hydrocarbon components provide a level of volatility that approximates the silicone compounds typically used in cosmetics, while the non-volatile component provides aesthetic properties similar to the replaced volatile silicone compounds.

In one embodiment, the volatile hydrocarbon components are straight or branched chain hydrocarbons or isoparaffins, such as isododecane. In another embodiment, the volatile hydrocarbon components are isoalkanes having different chain lengths. In such an embodiment, the first volatile hydrocarbon component has chains ranging from C12-C14, while the second volatile hydrocarbon component has chains ranging from C13-C16. Typically, the second volatile hydrocarbon component has chains that are longer than the first volatile hydrocarbon, though the inverse may be true or the chains may be of equal length. In an exemplary embodiment, the carrier fluid is comprised of from about 20 to about 80 weight percent, such as about 30 weight percent, of the first volatile hydrocarbon component, and from about 20 to about 80 weight percent, such as about 60 weight percent, of the second volatile hydrocarbon component.

The non-volatile hydrocarbon component may include “mixed” alkanes ranging from C13-C15 alkanes to C18-C21 alkanes and containing various amounts of cycloalkanes and n-alkanes as well as the branched chained isoparaffins or cosmetically acceptable oils. In an embodiment wherein the non-volatile hydrocarbon component is a mixed alkane with chains of different chemistries ranging from C13 to C21, the mixed alkane is used at concentrations from about 5 to about 20 weight percent, such as about 10 weight percent, of the carrier fluid.

The non-volatile hydrocarbon component may include an isohexadecane, isoalkanes such as a C13-C16 isoparaffin, straight chained alkanes such as a C13-C15 alkane, cycloalkanes, a mixture of isoalkanes and mixed structure hydrocarbons selected from linear, branched, and cyclohydrocarbons; and/or a C13-C21 straight or branched chain alkyl ester of a straight or branched chain carboxylic acid. The non-volatile hydrocarbon component may further include cosmetically acceptable oils such as isopropyl myristate, isopropyl palmitate, mineral oil, isononyl isononanoate, polybutenes, polyisobutenes and hydrogenated polyisobutenes.

In an exemplary embodiment, the carrier fluid is present in an amount of from about 25.0 to about 50.0 weight percent of the silicone-free antiperspirant composition. In another exemplary embodiment, the carrier fluid is composed of about 35 weight percent C12-C14 isoparaffin, about 55 weight percent C13-C16 isoparaffin, and about 10 weight percent C13-C15 alkane.

The silicone-free antiperspirant composition further includes stearyl alcohol. Stearyl alcohol is a fatty alcohol that serves as a structurant that provides, at least in part, the solid consistency of the silicone-free antiperspirant composition. In one exemplary embodiment, stearyl alcohol is present in the silicone-free antiperspirant composition in an amount of from about 15.0 to about 22.0 wt. % of the total weight of the antiperspirant composition. In this regard, in one embodiment, the stearyl alcohol, and other optional structurants as discussed below, is present in an amount sufficient to impart a hardness to the silicone-free antiperspirant composition in the range of from about 150 to about 350 grams-force, preferably in the range of from about 165 to about 215 grams-force, as measured by a TA.XT2i Texture Analyzer at the following settings: pre-speed—1.0 millimeters/second (mm/s); trigger force—5.0 grams (g); test speed—1.0 mm/s; retraction speed—5.0 mm/s; distance—5.0 mm; and cycles—1. The TA.XT2i is manufactured by Stable Micro Systems Ltd. of the United Kingdom.

Other structurants and/or gellants (hereinafter referred to collectively as “structurants”) that, along with stearyl alcohol, can facilitate the solid consistency of the silicone-free antiperspirant composition include naturally-occurring or synthetic waxy materials or combinations thereof. Suitable structurants, including waxes and gellants, are often selected from fatty alcohols containing from 12 to 30 carbons, such as behenyl alcohol and sterols such as lanosterol. As used herein, the term “fatty” means a long chain aliphatic group, such as at least 8 or 12 linear carbons, which is frequently not branched (linear) and is typically saturated, but which can alternatively be branched and/or unsaturated. It is possible for the fatty acid to contain a hydroxyl group, as in 12-hydroxystearic acid, for example as part of a gellant combination, and to employ amido or ester derivates thereof.

Other structurants can comprise hydrocarbon waxes such as paraffin waxes, microcrystalline waxes, ceresin, squalene, and polyethylene waxes. Other suitable structurants are waxes derived or obtained from plants or animals such as hydrogenated castor oil, hydrogenated soybean oil, carnabau, spermacetti, candelilla, beeswax, modified beeswaxes, and Montan wax and individual waxy components thereof. It is also suitable herein to employ a mixture of wax structurants. Suitable mixtures of structurants can reduce the visibility of active antiperspirant compounds deposited on the skin and result in either a soft solid or a firm solid.

The various embodiments of the silicone-free antiperspirant composition contemplated herein further comprise cetyl alcohol (comprising 16 carbons), myristyl alcohol (comprising 14 carbons), or a combination thereof (hereinafter, referred to collectively as a “C14-C16 fatty alcohol”). As noted above, based on information and belief, the C14-C16 fatty alcohols likely form a fatty alcohol matrix with stearyl alcohol that prevents the formation of crystals on the surface of the silicone-free antiperspirant composition upon manufacture thereof. In one exemplary embodiment, the C14-C16 fatty alcohols are present in an amount of from about 0.3 to about 6.0 wt. % of the silicone-free antiperspirant composition. In another exemplary embodiment, the C14-C16 fatty alcohols and the stearyl alcohol are present in a C14-C16 fatty alcohol:stearyl alcohol ratio of from about 1:2.5 to about 1:60, preferably 1:14.

In some embodiments, because of the low melting point of the C14-C16 fatty alcohols (about 49° C. for cetyl alcohol and about 37-39° C. for myristyl alcohol) compared to stearyl alcohol (about 60° C.), the addition of the C14-C16 fatty alcohols may cause the silicone-free antiperspirant composition to have a lower than desired melting point. While it is desirable for the silicone-free antiperspirant composition to slightly melt upon application to skin, it is also desirable for the silicone-free antiperspirant composition to be thermally stable and maintain a solid form at 45° C. Accordingly, in an exemplary embodiment, it may be desirable to include hydrogenated castor oil in the silicone-free antiperspirant composition in an amount in the range of from about 3.0 to about 10.0 wt. % of the antiperspirant compound. In another optional embodiment, it may be desirable to add high molecular weight (high MW) polyethylene to the antiperspirant compound. As used herein, the term “high molecular weight polyethylene” or “high MW polyethylene” means polyethylene having a molecular weight of from about 200 to about 5000 daltons (Da). High MW polyethylene has a melting point of about 70° C.-100° C. and can raise the melting point of the silicone-free antiperspirant composition. In one embodiment, the silicone-free antiperspirant composition comprises from about 1.0 to about 5.0 wt. % high MW polyethylene. In a preferred embodiment, the high MW polyethylene has a molecular weight of about 500 Da.

The antiperspirant compositions also may comprise a high refractive index (R.I.) hydrophobic compound serving as a residue masking agent. As used herein, the term “high refractive index” means a refractive index of no less than about 1.4. The high R.I. hydrophobic compound facilitates the minimization and/or prevention of a white residue on the skin by masking the active antiperspirant salt that stays upon the skin upon evaporation of the carrier, discussed in more detail below. Examples of high R.I. hydrophobic compounds for use in the antiperspirant products include PPG-14 butyl ether and C₁₂-C₁₅ alkyl benzoate, such as Finsolv TN® available from Innospec of the United Kingdom. In a preferred embodiment, the antiperspirant product comprises from about 10.0 to about 18.0 wt. % PPG-14 butyl ether.

In addition to the ingredients identified above, the silicone-free antiperspirant composition may comprise additives, such as those used in conventional antiperspirants. These additives include, but are not limited to, fragrances, including encapsulated fragrances, dyes, pigments, preservatives, antioxidants, moisturizers, and the like. These optional ingredients can be included in the silicone-free antiperspirant composition in an amount of 0 to about 20 wt. %. In a preferred embodiment, the silicone-free antiperspirant composition comprises from about 0.5 to about 2.5 wt. % fragrance. In another preferred embodiment, the silicon-free antiperspirant composition comprises from about 1.0 to about 6.0 wt. % aluminum starch octenylsuccinate or talc to provide a dry feel to the skin upon application.

The antiperspirant compositions, according to various embodiments, can be prepared by first combining the stearyl alcohol, the C14-C16 fatty alcohol, and the polyethylene, if used, and then melting them or, alternatively, melting each component and then mixing them, to form a melted mixture. The remaining ingredients, including the silicone-free carrier fluid can be added to the melted mixture, either separately or as one or more premixtures, to form a liquid active mixture. The active mixture is then poured into molds and permitted to cool at room temperature or with the assistance of a cooling chamber or cooling tunnel to hasten the solidification of the invisible solid antiperspirant product.

The following is an exemplary embodiment of a silicone-free antiperspirant composition, with each of the components set forth in weight percent of the silicone-free antiperspirant composition. The example is provided for illustration purposes only and is not meant to limit the various embodiments of the silicone-free antiperspirant composition in any way. All materials are set forth in weight percent.

### EXAMPLE 1

| Ingredient | Wt. % |
|---|---|
| SiClone ® SR5 | 32.57 |
| Aluminum zirconium | 22.03 |
| trichlorohydrex GLY | |
| Stearyl alcohol | 17 |
| Cetyl alcohol | .5 |
| Hydrogenated castor oil | 7.8 |
| PPG-14 butyl ether | 14 |
| Aluminum Starch | 3 |
| Octenylsuccinate | |
| Polyethylene | 2 |
| Fragrance | 1.1 |
| Total | 100.00 |

where SiClone® SR5 is a silicone-free carrier fluid available from Presperse LLC of Somerset, N.J. and composed of C12-C14 isoparaffin, C13-C16 isoparaffin, and C13-C15 alkane, and where Performalene 500 is polyethylene having a molecular weight of 500 Da.

The antiperspirant composition of Example 1 was manufactured by adding 880 grams (g) silicone-free carrier fluid to a vessel and initiating agitation. Utilizing high shear mixing, 859.48 g aluminum zirconium pentachlorohydrex GLY complex was incrementally added to form an active premix having a consistent fluid appearance without any particulates.

In another mixing container, 39 g hydrogenated castor oil, 85 g stearyl alcohol, 70 g PPG-14 butyl ether, 2.5 g cetyl alcohol, and 10 g Performalene 500 were added and heat was initiated to melt the components. Agitation was slowly initiated as the mixture became molten. The mixture was not permitted to exceed 99° C. When all of the components were molten, with continuous agitation, 252.5 g of the active premix was incrementally added while the batch temperature was maintained between 59° C. and 64° C. and mixing was continued until the mixture was homogeneous. The silicone-free antiperspirant composition then was cooled to 49° C., poured into a suitable mold and permitted to cool to ambient temperatures. As used herein, “permitted to cool to ambient temperatures” means either that the silicone-free antiperspirant composition is exposed to the ambient for a sufficient amount of time that it cools to the ambient temperature or that it is subjected to an artificial cooling means, such as a fan, refrigerator, or the like, that cools the silicone-free antiperspirant composition to ambient temperatures.

Accordingly, various embodiments relating to an antiperspirant composition that uses a carrier fluid composed of hydrocarbons rather than volatile silicone are provided. The silicone-free antiperspirant includes stearyl alcohol as a desired structurant yet does not exhibit surface crystals upon manufacture. In this regard, the various embodiments comprise a C14-C16 fatty alcohol. As noted above, while stearyl alcohol is a desired structurant in antiperspirant compositions because of its ability to impart structure and hardness to the compositions, in certain compositions it may cause crystals to form at the surface of the compositions after manufacture. It is believed that the C14-C16 fatty alcohol present in sufficient amounts in the silicone-free antiperspirant composition, forms a fatty alcohol matrix with the stearyl alcohol that prevents the crystals from forming.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the claimed subject matter in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope defined by the claims, which includes known equivalents and foreseeable equivalents at the time of filing this patent application.

1. A silicone-free antiperspirant composition comprising:
an active antiperspirant compound;
stearyl alcohol;
a C14-C16 fatty alcohol; and
a carrier fluid composed of a first volatile hydrocarbon component that is a C12-C14 isoparaffin, a second volatile hydrocarbon component that is a C13-C16 isoparaffin, and a non-volatile hydrocarbon component that is a C13-C15 alkane, wherein the carrier fluid is present in an amount of from about 25.0 to about 50.0 weight percent of the total weight of the silicone-free antiperspirant composition.

2. The silicone-free antiperspirant composition of claim 1 wherein the C14-C16 fatty alcohol is cetyl alcohol.

3. The silicone-free antiperspirant composition of claim 1 wherein the C14-C16 fatty alcohol is present in an amount of from about 0.3 to about 6.0 weight percent of the silicone-free antiperspirant composition.

4. The silicone-free antiperspirant composition of claim 1 wherein the C14-C16 fatty alcohol is present in a C14-C16 fatty alcohol:stearyl alcohol ratio of from about 1:2.5 to about 1:60.

5. The silicone-free antiperspirant composition of claim 1 further comprising a high molecular weight polyethylene in an amount of from about 1.0 to about 5.0 weight percent of the total weight of the silicone-free antiperspirant composition.

6. The silicone-free antiperspirant composition of claim 1 further comprising hydrogenated castor oil in an amount of from about 3.0 to about 10.0 weight percent of the total weight of the silicone-free antiperspirant composition.

7. The silicone-free antiperspirant composition of claim 1 further comprising a residue masking agent in an amount of from about 8.0 to about 18.0 weight percent of the total weight of the silicone-free antiperspirant composition.

8. The silicone-free antiperspirant composition of claim 1 further comprising aluminum starch octenylsuccinate or talc in an amount of from about 1.0 to about 6.0 weight percent of the total weight of the silicone-free antiperspirant composition.

9. (canceled)

10. The silicone-free antiperspirant composition of claim 9 wherein the carrier fluid is comprised of about 35 weight percent C12-C14 isoparaffin, about 55 weight percent C13-C16 isoparaffin, and about 10 weight percent C13-C15 alkane.

11. (canceled)

12. The silicone-free antiperspirant composition of claim 1 wherein at least one of the volatile hydrocarbon components is chosen from the group consisting of a straight or branched chain hydrocarbon, an isoparaffin, isododecane, and a C12-C14 isoparaffin.

13. The silicone-free antiperspirant composition of claim 12 wherein the non-volatile hydrocarbon component is chosen from the group consisting of a C13-C21 hydrocarbon; isohexadecane; a mixture of isoalkanes and mixed structure hydrocarbons selected from linear, branched, and cyclohydrocarbons; a C13-C15 alkane; a C13-C16 isoparaffin; a C13-C21 straight or branched chain alkyl ester of a straight or branched chain carboxylic acid; and mineral oil.

14. An antiperspirant composition comprising:
an active antiperspirant compound;
stearyl alcohol present in an amount of from about 15.0 to about 22.0 weight percent of the total weight of the antiperspirant composition;
cetyl alcohol present in an amount of from about 0.3 to about 6.0 weight percent of the total weight of the antiperspirant composition;
a silicone-free carrier fluid present in an amount of from about 25.0 to about 50.0 weight percent of the composition, wherein the silicone-free carrier fluid is composed of a first volatile hydrocarbon component selected from a C12-C14 hydrocarbon, a second volatile hydrocarbon component selected from a C13-C16 hydrocarbon, and a non-volatile hydrocarbon component,
hydrogenated castor oil in an amount of from about 3.0 to about 10.0 weight percent of the total weight of the antiperspirant composition;
a residue masking agent in an amount of from about 8.0 to about 18.0 weight percent of the composition;
polyethylene in an amount of from about 1.0 to about 5.0 weight percent of the composition; and
aluminum starch octenylsuccinate or talc in an amount of from about 1.0 to about 6.0 weight percent of the composition.

15. The antiperspirant composition of claim 14 wherein the first volatile hydrocarbon component is a C12-C14 isoparaffin, the second volatile hydrocarbon component is a C13-C16 isoparaffin, and the non-volatile hydrocarbon component is a C13 -C15 alkane.

16. The antiperspirant composition of claim 14 wherein the silicone-free carrier fluid is comprised of about 35 weight percent C12-C14 isoparaffin, about 55 weight percent C13-C16 isoparaffin, and about 10 weight percent C13-C15 alkane of the total weight of the antiperspirant composition.

17. The antiperspirant composition of claim 14 wherein at least one of the volatile hydrocarbon components is chosen from the group consisting of a straight or branched chain hydrocarbon, an isoparaffin, isododecane, and a C12-C14 isoparaffin.

18. The antiperspirant composition of claim 17 wherein the non-volatile hydrocarbon component is chosen from the group consisting of a C13-C21 hydrocarbon; isohexadecane; a mixture of isoalkanes and mixed structure hydrocarbons selected from linear, branched, and cyclohydrocarbons; a C13-C15 alkane; a C13-C16 isoparaffin; a C13-C21 straight or branched chain alkyl ester of a straight or branched chain carboxylic acid; and mineral oil.

19. (canceled)

20. A method for fabricating a silicone-free antiperspirant composition, the method comprising the steps of:
combining a C14-C16 fatty alcohol and stearyl alcohol in a C14-C16 fatty alcohol:stearyl alcohol ratio of from about 1:2.5 to about 1:60;
melting the stearyl alcohol and the C14-C16 fatty alcohol;
adding a carrier fluid to the stearyl alcohol and the C14-C16 fatty alcohol, wherein the carrier fluid is composed of a first volatile hydrocarbon component selected from a C12-C14 hydrocarbon, a second volatile hydrocarbon component selected from a C13-C16 hydrocarbon, and a non-volatile hydrocarbon component;
fowling a melted mixture;
pouring the melted mixture into a mold; and
allowing the melted mixture to cool to ambient temperature.
